(19) United States
(12) Patent Application Publication
(10) Pub. No.: US 2014/0335062 A1
(43) Pub. Date: Nov. 13, 2014

(54) CO-CULTURED MESENCHYMAL STEM CELLS AND MYOCYTES

(71) Applicant: University of Miami, Miami, FL (US)

(72) Inventor: Joshua M. Hare, Miami Beach, FL (US)

(21) Appl. No.: 14/372,850

(22) PCT Filed: Jan. 18, 2013
(86) PCT No.: PCT/US2013/022271

§ 371 Date: Jul. 17, 2014

Related U.S. Application Data
(60) Provisional application No. 61/589,059, filed on Jan. 20, 2012.

(51) Int. Cl. C12N 5/077; A61K 35/28; A61K 35/34

(52) U.S. Cl. CPC C12N 5/0657; A61K 35/34; A61K 35/28; C12N 2506/1346; C12N 2502/1352; C12N 2513/00
USPC 424/93.7; 435/373; 435/347

(57) ABSTRACT

Co-culturing of mesenchymal stem cells or tissues comprising mesenchymal stem cells with myocytes or myocyte containing tissues provide for the long term culture and expansion of myocytes. The co-culture gives rise to three-dimensional functioning cardiac tissue grafts or constructs.

### STATEMENT OF FEDERAL FUNDING

This invention was made with federal funds by the National Institutes of Health, grant number RO1 AG025017. The U.S. government may have certain rights.

### FIELD OF INVENTION

Embodiments of the invention are directed to tissue engineering for use in drug screening and therapeutic applications, in particular, to long term cultures of excitable tissues tor development of biological agents, especially; three-dimensional tissue constructs, and maintenance of stem cells.

### BACKGROUND

A central challenge for research in regenerative medicine is to develop cell compositions that can help reconstitute cardiac function. It is estimated that nearly one in five men and women have some form of cardiovascular disease (National Health and Nutrition Examination Survey III, 1988-94, Center of Disease Control and the American Heart Association). Widespread conditions include coronary heart disease (5% of the population), congenital cardiovascular defects (0.5%), and congestive heart failure (3%). The pharmaceutical arts have produced small molecule drugs and biological compounds that can help limit the damage that occurs as a result of heart disease, but there is nothing commercially available to help regenerate the damaged tissue.

### SUMMARY

This Summary is provided to present a summary of the invention to briefly indicate the nature and substance of the invention. It is submitted with the understanding that it will not be used to interpret or limit the scope or meaning of the claims.

Embodiments of the invention are directed, inter alia, to co-culture systems of mesenchymal stem cells and myocytes. The myocytes can originate from cardiac stem cells, neonatal myocytes or adult myocytes. The co-culturing yields stable and durable beating constructs of myocardial tissue. These constructs can be maintained in culture for months, and form 3-dimensional tissue constructs that beat spontaneously.

In one embodiment, constructs of tissue are used for drug screening to measure excitation contraction coupling. In addition, the ability of MSCs to enhance growth and provide stability to myocyte cells provides an opportunity to employ tissues from genetically affected patients so as to create a 3-dimensional tissue construct specific to an Individual or groups of individuals.

In other embodiments, the tissue constructs are used for therapies requiring tissue engineered material. Examples, include, without limitation: would be congenital heart disease, heart failure, MI, LVD, or any cardiac disease or disorder which damage tissues.

In another embodiment, a composition comprises ex-vivo functional cardiac cells and tissues. In some aspects the constructs comprise tissue engineered functional myocardium.

Other aspects of the invention are described infra.

### BRIEF DESCRIPTION OF THE DRAWINGS

FIG. 1A and 1B is a scan of photographs from a fluorescent microscope showing that human MSCs (GFP⁺) in co-culture with NRVMs form beating tubes. GFP⁺cells integrate into tube.

FIG. 2 is a scan of a photograph from a fluorescent microscope showing a beating sphere in co-culture CX43 knockdown hMSCs with NRVMs.

FIG. 3A is a scan of a photograph showing that beating tube formation occurred, after 10 days in co-culture NRVMs with hMSCs overexpressing Cx43, FIG. 3B shows the control group where tube formation occurred after 1-4 days in co-culture.

FIG. 4A is a scan of a photograph showing that in co-cultured hMSCs Cx43 knockdown with NRVMs beating tube formation never occurred. FIG. 48 shows that in the control group beating tube formation occurred after 14 days in co-culture.

FIG. 5A is a scan of a photograph showing that beating spheres occurred in NRVMs with hMSC Cx43 knockdown co-culture after 4 weeks. FIG. 5B is a scan of a photograph showing that NRVMs alone form beating spheres in culture.

FIG. 6 is a graph showing the percentage of tube formation at 14 days.

FIG. 7 is a scan of a photograph from a fluorescent microscope showing beating tube formation of NRVMs with human MSCs overexpressing Cx43.

FIG. 8 is a scan of a photograph showing beating spheres in co-culture NRVMs with human MSCs Cx43 knockdown.

### DETAILED DESCRIPTION

The present invention is described with reference to the attached figures, wherein like reference numerals are used throughout the figures to designate similar or equivalent elements. The figures are not drawn to scale and they are provided merely to illustrate the instant invention. Several aspects of the invention are described below with reference to example applications for illustration, it should be understood that numerous specific details, relationships, and methods are set forth to provide a full understanding of the invention. One having ordinary skill in the relevant art, however, will readily recognize that the invention can be practiced without one or more of the specific details or with other methods. The present invention is not limited by the illustrated ordering of acts or events, as some acts may occur in different orders and/or concurrently with other acts or events. Furthermore, not all illustrated, acts or events are required to implement a methodology in accordance with the present invention.

Embodiments of the invention may be practiced without the theoretical aspects presented. Moreover, the theoretical aspects are presented with the understanding that Applicants do not seek to be bound by the theory presented.

Unless otherwise defined, all terms (including technical and scientific terms) used herein have the same meaning as commonly understood by one of ordinary skill in the art to which this invention belongs. It will he further understood that terms, such as those defined in commonly used dictionaries, should be interpreted as having a meaning that is consistent with their meaning in the context of the relevant art and will not be interpreted in an idealized or overly formal sense unless expressly so defined herein.

### Definitions

The terminology used herein is for the purpose of describing particular embodiments only and is not intended to be limiting ox the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to he inclusive in a manner similar to the terra, “comprising.”

The term “about” or “approximately” means within an acceptable error range for the particular value as determined by one of ordinary skill in the art, which will depend in part, on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively,, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

A “stem cell” is a cell from the embryo, fetus, or adult that has under certain conditions, the ability to reproduce itself for long periods or, in the case of adult stem cells, throughout the life of the organism. It also can give rise to specialized cells that make up the tissues and organs of the body. A “pluripotent stem cell” has the ability to give rise to types of cells that develop from the three germ layers (mesoderm, endoderm, and ectoderm) from which all the cells of the body arise. The only known sources of human pluripotent stem cells are those isolated and cultured from early human embryos and from fetal tissue that was destined to be part of the gonads. An “embryonic stem cell” is derived from a group of cells called the inner cell, mass, which is part of the early (4- to 5-day) embryo called the blastocyst. Once removed from the blastocyst the cells of the inner cell mass can be cultured into embryonic stem cells. These embryonic stem cells are not themselves embryos. An “adult stem cell” is an undifferentiated (unspecialized) cell that occurs in a differentiated (specialized) tissue, renews itself, and becomes specialized to yield all of the specialized cell types of the tissue in which it is placed when transferred to the appropriate tissue. Adult stem cells are capable of making identical copies of themselves for the lifetime of the organism. This property is referred to as “self-renewal.” Adult stem cells usually divide to generate progenitor or precursor cells, which then differentiate or develop into “mate” cell types that have characteristic shapes and specialized functions, e.g., muscle cell contraction or nerve cell signaling. Sources of adult stem cells include hone marrow, blood, the cornea and the retina of the eye, brain, skeletal muscle, dental pulp, liver, skin, the lining of the gastrointestinal tract and pancreas.

A “progenitor or precursor” cell occurs in fetal or adult tissues and is partially specialized; it divides and gives rise to differentiated cells. Researchers often distinguish precursor/progenitor cells from adult, stem cells in that when a stem cell divides; one of the two new cells is often a stem cell capable of replicating itself again, in contrast when a progenitor/precursor cell divides, it can form more progenitor/precursor cells or it can form two specialized cells. Progenitor/precursor cells can replace cells that are damaged or dead, thus maintaining the integrity and functions of a tissue such as liver or brain.

As used herein, “heart disease” or “cardiac disease” refers to any type of heart disease including cardiomyopathy, hypertrophic cardiomyopathy, dilated cardiomyopathy, atherosclerosis, coronary artery disease, ischemic heart disease, myocarditis, viral infection, wounds, hypertensive heart disease, valvular disease, congenital heart disease, myocardial infarction, congestive heart failure, arrhythmias, diseases resulting in remodeling of the heart, etc. Diseases of the heart can be due to any reason, such as for example, damage to cardiac tissue such as a loss of contractility (e.g., as might be demonstrated by a decreased ejection traction).

“Cardiac damage” or “cardiac disorder” characterized by insufficient cardiac function includes any impairment or absence of a normal cardiac function or presence of an abnormal cardiac function. Abnormal cardiac function can be the result of disease, injury, and/or aging. As used herein, abnormal cardiac function includes morphological and/or functional abnormality of a cardiomyocyte, a population of cardiomyocytes, or the heart itself. Non-limiting examples of morphological and functional abnormalities include physical deterioration and/or death of cardiomyocytes, abnormal growth patterns of cardiomyocytes, abnormalities in the physical connection between cardiomyocytes, under- or over-production of a substance or substances by cardiomyocytes, failure of cardiomyocytes to produce a substance or substances which they normally produce, and transmission of electrical impulses in abnormal patterns or at abnormal times. Abnormalities at a more gross level include dyskinesis, reduced ejection fraction, changes as observed by echocardiography (e.g., dilatation), changes in EKG, changes in exercise tolerance, reduced capillary perfusion, and changes as observed by angiography. Abnormal cardiac function is seen with many disorders including, for example, ischemic heart disease, e.g., angina pectoris, myocardial infarction, chronic ischemic heart disease, hypertensive heart disease, pulmonary heart disease (cor pulmonale), valvular heart disease, e.g., rheumatic fever, mitral valve prolapse, calcification of mitral annulus, carcinoid heart disease, infective endocarditis, congenital heart disease, myocardial disease, e.g., myocarditis, dilated cardiomyopathy, hypertensive cardiomyopathy, cardiac disorders which result in congestive heart failure, and tumors of the heart, e.g., primary sarcomas and secondary tumors. Heart damage also includes wounds, such as for example, knife wound; biological (e.g. viral; autoimmune diseases) or chemical (e.g. chemotherapy, drugs): surgery; transplantation and the like.

“Myocardial ischemia” refers to a lack of oxygen flow to the heart which results in myocardial ischemic damage. As used herein, the phrase myocardial ischemic damage includes damage caused by reduced blood flow to the myocardium. Non-limiting examples of causes of myocardial ischemia and myocardial ischemic damage include: decreased aortic diastolic pressure, increased intraventricular pressure and myocardial contraction, coronary artery stenosis (e.g., coronary ligation, fixed coronary stenosis, acute plaque change (e.g., rupture, hemorrhage), coronary artery thrombosis, vasoconstriction), aortic valve stenosis and regurgitation, and increased right atrial pressure. Non-limiting examples of adverse effects of myocardial ischemia and myocardial ischemic damage include; myocyte damage (e.g., myocyte cell loss, myocyte hypertrophy, myocyte cellular hyperplasia), angina (e.g., stable angina, variant angina, unstable angina, sudden cardiac death), myocardial infarction, and congestive heart fail ore. Damage due to myocardial ischemia may be acute or chronic, and consequences may include sear formation, cardiac remodeling, cardiac hypertrophy, wall thinning, dilatation, and associated functional changes. The existence and etiology of acute or chronic myocardial damage and/or myocardial ischemia may be diagnosed using any of a variety of methods and techniques well known in the art including, e.g., non-invasive imaging (e.g., MRI, echocardiography), angiography, stress testing, assays for cardiac-specific proteins such as cardiac troponin, and clinical symptoms. These methods and techniques as well as other appropriate techniques may be used to determine which subjects are suitable candidates for the treatment methods described herein.

As used herein, “electrical coupling” means the interaction between cells which allows for intracellular communication between cells so as to provide for electrical conduction between the cells. Electrical coupling m vivo provides the basis for, and is generally accompanied by, electromechanical coupling, in which electrical excitation of cells through gap junctions in the muscle leads to muscle contraction.

“Biological samples” include solid and body fluid samples. The biological samples used in the present invention can include cells, protein or membrane extracts of cells, blood or biological fluids such, as ascites fluid or brain fluid (e.g., cerebrospinal fluid). Examples of solid biological samples include, but are not limited to, samples taken from tissues of the central nervous system, bone, breast, kidney, cervix, endometrium, head/neck, gallbladder, parotid gland, prostate, pituitary gland, muscle, esophagus, stomach, small intestine, colon, liver, spleen, pancreas, thyroid, heart, lung, bladder, adipose, lymph node, uterus, ovary, adrenal gland, testes, tonsils and thymus. Examples of “body fluid samples” include, but are not limited to blood, serum, semen, prostate fluid, seminal fluid, urine, saliva, sputum, mucus, bone marrow, lymph, and tears.

As used herein, the term “autologous” is meant to refer to any material derived from the same individual to whom it is later to be reintroduced into the individual.

The term “xenogeneic” refers to a cell or tissue that derives from a different animal species than the animal species that becomes the recipient animal host in a transplantation or vaccination procedure.

The term “allogeneic” refers to a cell or tissue that is of the same animal species but genetically different in one or more genetic loci as the animal that becomes the “recipient, host”. This usually applies to cells or tissues transplanted from one animal to another non-identical animal of the same species.

The term “syngeneic” refers to a cell or tissue which is of the same animal species and has the same genetic composition for most genotypic and phenotypic markers as the animal who becomes the recipient host of that cell line in a transplantation or vaccination procedure. This usually applies to cells or tissue transplanted from identical twins or may be applied to cells transplanted between highly inbred animals.

The terms “patient” or “individual” are used Interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters; and primates.

“Diagnostic” or “diagnosed” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives,” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

“Treatment” is an intervention performed with the intention of preventing the development or altering the pathology or symptoms of a disorder. Accordingly, “treatment” refers to both therapeutic treatment and prophylactic or preventative measures. Those in need of treatment include those already with the disorder as well as those in which the disorder is to be prevented. As used herein, “ameliorated” or “treatment” refers to a symptom which is approaches a normalized value (for example a value obtained in a healthy patient or individual), e.g., is less than 30% different from a normalized value, preferably is less than about 25% different from a normalized value, more preferably, is less than 10% different from a normalized value, and still more preferably, is not significantly different from a normalized value as determined using routine statistical tests.

### General Techniques

For further elaboration of general techniques useful in the practice of this invention, the practitioner can refer to standard textbooks and reviews In cell biology, tissue culture, embryology, and cardiophyslology.

Methods involving conventional molecular biology techniques are described herein. Such techniques are generally known in the art and are described in detail in methodology treatises such as Molecular Cloning: Laboratory Manual, 2nd ed. vol. 1-3. ed. Sambrook et al., Cold Spring Harbor Laboratory Press, Cold Spring Harbor, N.Y., 1989; and Current Protocols in Molecular Biology, Ausobel et al. eds., (with periodic updates). Various techniques using polymerase chain reaction (PCR) are described, for example, in Innis et al., PCR Protocols: A Guide to Methods and Applications, Academic Press: San Diego, 1990. PCR primer pairs can be derived thorn known sequences by known techniques, such as use of computer programs intended for that purpose (for example, Primer, Versions 0.5,1991, Whitehead Institute for Biomedical Research, Cambridge, Mass.). Methods for chemical synthesis of nucleic acids are discussed, for example, in Beaucage and Camuhers, Tetra Letts 22; 1859-1862, 1981, and Matteucci et al., J Am Chem Soc 103:3185,1981. Chemical synthesis of nucleic acids can be performed, for example, on commercial automated oligonucleotide synthesizers.

With respect to tissue culture and embryonic stem cells, the reader may wish to refer to Teratocarcinomas and embryonic stem cells; A practical approach (E. J. Robertson, ed., IRL Press Ltd. 1987); Guide to Techniques in. Mouse Development (P. M. Wasserman et al. eds,, Academic Press 1993): Embryonic Stem Cell Differentiation in Vitro (M. V. Wiles, Meth. Enzymol. 225:900,1993); Properties and uses of Embryonic Stem Cells: Prospects for Application to Human Biology and Gene Therapy (P. D. Rathjen et al., Reprod. Fertil. Dev. 10:31, 1998). With respect to the culture of heart cells, standard references include The Heart Cell in Culture (A. Pinson ed., CRC Press 1987), Isolated Adult Cardiomyocytes (Vols. I & II, Piper & isenberg eds., CRC Press 1989), Heart Development (Harvey & Rosenthal, Academic Press 1998), I Left my Heart in San Francisco (T. Bonnet, Sony Records 1990): and Gone with the Wnt (M. Mitchell, Scribner 1996).

Immunological methods (for example, preparation of antigen-specific antibodies, immunoprecipitation and immunoblotiing and immunolocalization) are described, for example, in Current Protocols in Immunology, Coligan et al., eds., John Wiley & Sons, New York, 1991; and Methods of Immunological Analysis, Masseyeff et al., eds., John Wiley & Sons, New York, 1992.

Conventional methods of gene transfer and gene therapy can also be adapted for use in the present invention. See, for example. Gene Therapy: Principles and Applications T Blackenstein, ed., Springer Verlag, 1999; Gene Therapy Protocols (Methods in Molecular Medicine), P D Robbins ed., Humana Press, 1997; and Retro-vectors for Human Gene Therapy, C P Hodgson, ed., Springer Verlag, 1996.

### Tissue Constructs and Cellular Compositions

There is a major need for ex vivo functional cardiac cells and tissues. The inability to maintain adult myocytes alive ex vivo has been a major limitation in drug, development. Also constructs for tissue engineered functional myocardium are limited.

Embodiments of the invention are directed to co-culture systems of mesenchymal stem cells and myocytes. The co-culturing yields stable and durable beating constructs of myocardial tissue. These constructs can be maintained in culture for months, and form 3-dimensional tissue constructs that beat spontaneously. These constructs can be used for drug screening to measure excitation contraction coupling. The ability of MSCs to enhance growth and provide stability to myocyte cells provides an opportunity to employ tissues from genetically affected patients so as to create a three-dimensional tissue construct specific to an individual or groups of individuals. The constructs could also be used for therapies requiring tissue engineered material. Examples would be congenital heart disease, heart failure, MI, LVD.

In embodiments, a composition comprises mesenchymal stem cells (MSCs), myocytes and/or three dimensional heating tissues (3D-tissues). In some embodiments, the composition comprises tissues comprising mesenchymal cells and/or myocytes. Once the myocytes are cultured and expanded they can be isolated and purified and maintained in culture for use in vivo or for in vitro assays, such as for example, diagnostic assays, drug discovery etc.

In another embodiment, a co-culture system comprises isolated mesenchymal stem cells and/or tissues comprising mesenchymal stem cells, myocytes and/or tissues comprising myocytes or combinations thereof. The mesenchymal stem cells, myocytes or tissues can be derived from various sources comprising autologous, heterologous, syngeneic, allogeneic or xenogeneic cells or tissues. In embodiments, the mesenchymal stem cells are multi-lineage stem cells. In another embodiment, the myocytes comprise cardiac stem cells, neonatal myocytes, adult myocytes or combinations thereof. In another embodiment, the co-culture system comprises a tissue for the co-culture of mesenchymal stem cells and myocytes. The tissue can be from any source including donor derived tissues.

In embodiments, the co-culture of mesenchymal stem cells and myocytes differentiate and form a three-dimensional beating myocardial tissue,

In another preferred embodiment, a method of engineering a three-dimensional tissue comprises co-culturing an effective amount of isolated mesenchymal stem cells and myocytes. The three-dimensional tissue is a functioning cardiac beating tissue. As discussed the mesenchymal stem cells and myocytes are autologous, heterologous, syngeneic, allogeneic or xenogeneic cells.

In another embodiment, the myocytes comprise cardiac stem cells, neonatal myocytes, adult myocytes or combinations thereof.

In another preferred embodiment, a method of culturing adult myocytes comprises: co-culturing an effective amount of isolated mesenchymal stem cells, in some embodiments, the co-culture comprises tissues comprising myocytes and/or mesenchymal stem cells. In some embodiments the co-culture comprises a layer of tissue for co-culturing mesenchymal stem and myocytes. The mesenchymal stem cells, myocytes or tissues are autologous, heterologous, syngeneic, allogeneic or xenogeneic cells or tissues.

In another preferred embodiment, a method of treating or repairing damaged cardiac tissue comprises administering to a patient in need thereof, myocytes which have been cultured ex vivo and/or grafted into a patient In need thereof a three-dimensional beating tissue graft in preferred embodiments, the cultured myocytes are obtained from a co-culture of mesenchymal stem cells and myocyte cells, in other preferred embodiments, the three-dimensional tissue beating construct is obtained by co-culturing an effective amount of isolated mesenchymal stem cells and myocytes.

In preferred embodiments the mesenchymal stem cells comprise connexin molecules. The molecules can be nucleic acids, peptides, polypeptides, proteins, mutants, variants or combinations thereof.

In one embodiment, the mesenchymal stem cells have been modified to over-express gap junction protein connexin 43 (Cx43). This results in the formation of spontaneously beating tubules of myocytes.

In another embodiment, the mesenchymal stem cells have been modified to knock-down expression of gap junction protein connexin 43. This results in the formation of spontaneously beating spheres of myocytes.

A further aspect of the subject matter is a lentivirus engineered to modify the mesenchymal stem cells to over-express gap junction protein connexin 43. The DNA sequence encoding the lentivirus can include Cx43, fragments, mutants, variants, or combinations thereof.

A further aspect of the subject matter is a lentivirus used to modify the mesenchymal stem cells to knock down expression of gap junction protein connexin 43.

A further aspect of the subject matter is a method for creating constructs of three-dimensional ex-vivo myocardial tissue. The method includes culturing myocytes together with mesenchymal stem cells containing a mutant connexin 43 gene to cause the formation of three-dimensional constructs of myocardial tissue.

In one embodiment, a method for treating cardiac disease in a host comprises (a) introducing a therapeutically effective amount of host cells co-cultured with mesenchymal stem cells containing a connexin 43 gene, mutants, fragments or variants thereof into the tissue of the host, and (b) thereby treating the cardiac disease in the host.

Mesenchymal Stem Cells: Mesenchymal stem cells are the formative pluripotential blast cells found inter alia in hone marrow, blood, dermis and periosteum that are capable of differentiating into any of the specific types of mesenchymal or connective tissues (i.e. the tissues of the body that support the specialized elements; particularly adipose, osseous, cartilaginous, elastic, and fibrous connective tissues) depending upon various influences from bioactive factors, such as cytokines.

In one preferred embodiment, mesenchymal stem cells are isolated from bone marrow of adult patients. In one aspect, the cells are passed through a density gradient to eliminate undesired cell types. The cells are preferably, plated and cultured in appropriate media. In another preferred embodiment, the cells are cultured for at least one day, preferably, about three to about seven days, and removing non-adherent cells. The adherent cells are plated and expanded.

Other means for isolating and culturing stem cells useful in the present invention are well known. Umbilical cord blood is an abundant source of hematopoietic stem cells. The stem cells obtained from umbilical cord blood and those obtained from bone marrow or peripheral blood appear to be very similar for transplantation use. Placenta is an excellent readily available source for mesenchymal stem cells. Moreover, mesenchymal stem cells can be derivable from adipose tissue and bone marrow stromal cells and speculated to be present in other tissues. While there are dramatic qualitative and quantitative differences in the organs from which adult stem cells can be derived, the initial differences between the cells may be relatively superficial and balanced by the similar range of plasticity they exhibit.

Homogeneous human mesenchymal, stem cell compositions are provided which serve as the progenitors for all mesenchymal cell lineages. MSCs are identified by specific cell surface markers which are identified with unique monoclonal antibodies. The homogeneous MSG compositions are obtained by positive selection of adherent marrow or periosteal cells which are tree of markers associated with either hematopoietic cell or differentiated mesenchymal cells. These isolated mesenchymal cell populations display epitopic characteristics associated with only mesenchymal stem cells, have the ability to regenerate in culture without differentiating, and have the ability to differentiate into specific mesenchymal lineages when either induced in vitro or placed in vivo at the site of damaged, tissue.

In order to obtain subject human mesenchymal stem cells, pluripotent mesenchymal stem cells are separated from other cells in the bone marrow or other MSG source. Bone marrow cells may be obtained from iliac crest, femora, tibiae, spine, rib or other medullary spaces. Other sources of human mesenchymal stem cells include embryonic yolk sac, placenta, umbilical cord, fetal and adolescent skin, and blood.

As discussed above, the mesenchymal stem cells can be isolated and purified by different methods. Other methods include, providing a tissue specimen containing mesenchymal stem cells, adding cells from the tissue specimen to a medium which contains factors that stimulate mesenchymal stem cell growth without differentiation and allows, when cultured, for the selective adherence of only the mesenchymal stem cells to a substrate surface, culturing the specimen-medium mixture, and removing the non-adherent matter from the substrate surface.

In a preferred embodiment, the mesenchymal stem cells are derived from, one or sources comprising: autologous, heterologous, syngeneic, allogeneic or xenogeneic sources. These sources can include cell lines. As used herein, “source” refers to the animal, in which these stem cells were obtained from, including human.

Differentiation of mesenchymal stem cells to the cardiac lineage is controlled by factors present in the cardiac environment. Local chemical, electrical and mechanical environmental influences alter pluripotent MSCs and convert: the cells administered to the heart into the cardiac lineage.

In a preferred embodiment, mesenchymal stem cells differentiate into lineages of cells that make up the different heart tissues. In some embodiments, the lineages are identified by cardiac cell specific markers comprising cardiac transcription factor GATA-4, MDR1; endothelial cell markers Factor VIII and KDR; vascular smooth muscle marker α-smooth muscle aclin; or eardiomyocyte marker α-sarcomerie actinin. For example, detection of expression of cardiomyoeyte specific proteins is achieved using antibodies specific for, for example, myosin heavy chain monoclonal antibody MF 20 (MF20), sarcoplasmic reticulum calcium ATPase (SERCA1) (mnAb 10D1) or gap junctions using antibodies to connexin 43, Other markers for cardiomyocytes comprise cardiac troponin I (c-TnI), cardiac troponin T (cTuT), sarcomeric myosin heavy chain (MHC), GATA-4, Nkx2.5, N-cadherin, β1-adrenoceptor β1-AR), ANF, the MEF-2 family of transcription factors, creatine kinase MB (CK-MB), myoglobin, or atrial natriuretic factor (ANF).

GATA transcription factor includes members of the GATA family of zinc finger transcription factors. GATA transcription factors are involved in the development of several mesodermally derived cell lineages. Preferably, GATA transcription factors include GATA-4 and/or GATA-6, The GATA-6 and GATA-4 proteins share high-level amino acid sequence identify over a proline-rich region at the amino terminus of the protein that is not conserved in other GATA family members.

In another preferred embodiment, soluble factors from mesenchymal, stem cell and myocyte cultures are administered to a patient with heart disease or disorders thereof. The factors are administered In an effective amount resulting in the localization, proliferation and maturation of cardiac stem cells Into cells of the damaged heart tissue.

In a preferred embodiment, cardiac stem cells are identified by markers comprising: c-kit^{pos}, CD3^{neg}, CD14^{neg} and CD68^{neg}.

Therapeutic Applications: Cardiac injury promotes tissue responses which enhance myogenesis using implanted myocytes, MSCs or three-dimensional beating tissue (3D-tissues) constructs. Thus, these compositions are introduced to the Infarct zone to reduce the degree of scar formation and to augment ventricular function. New muscle is thereby created within an infarcted myocardial segment Timing of intervention is designed to mimic the clinical setting where patients with acute myocardial infarction would first come to medical attention, receive first-line therapy, followed by stabilization, and then intervention with myocardial replacement therapy if necessary.

Of the four chambers of the heart, the left ventricle is primarily responsible for pumping blood under pressure through the body's circulatory system. It has the thickest myocardial walls and is the most frequent site of myocardial injury resulting from congestive heart failure. The degree of advance or severity of the congestive heart failure ranges from those cases where heart transplantation is indicated as soon as a suitable donor organ becomes available to those where little or no permanent injury is observed and treatment is primarily prophylactic.

The severity of resulting myocardial infarction, i.e. the percentage of muscle mass of the left ventricle that is involved can range from about 1 to about 80 percent. This represents affected tissue areas, whether as one contiguous ischemia or the sum of smaller ischemic lesions, having horizontal affected areas from about 1 cm² to about 6 cm² and a thickness of from 1-2 mm to 1-1.5 cm. The seventy of the infarction is significantly affected by which vessel(s) is involved and how much time has passed before treatment intervention is begun.

The compositions used in accordance with the invention are, in order of preference, autologous, allogeneic or xenogeneic, and the choice can largely depend on the urgency of the need for treatment. A patient presenting an imminently life threatening condition may be maintained on a heart/lung machine while sufficient numbers of autologous myocytes, tissues MSCs are cultured or initial treatment can be provided using other than autologous compositions.

The therapy of the invention can be provided by several routes of administration, including the following. First, intracardiac muscle injection, which avoids the need for an open surgical procedure, can be used where the cells or tissues are in an injectable liquid suspension preparation or where they are in a biocompatible medium which is injectable in liquid form and becomes semi-solid at the site of damaged myocardium. A conventional, intracardiac syringe or a controllable arthroscopic delivery device can be used so long as the needle lumen or bore is of sufficient diameter (e.g. 30 gauge or larger) that shear forces will not damage the cells or 3D-tissues. The injectable liquid suspension MSG preparations can also be administered intravenously, either by continuous drip or as a bolus. During open surgical procedures, involving direct physical access to the heat, all of the described forms of MSG delivery preparations are available options.

As a representative example of a dose range is a volume of about 20 to about 50 μl of injectable suspension containing about 10-40×10⁶ MSCs/ml. The concentration of cells per unit volume, whether the carrier medium is liquid or solid remains within substantially the same range. The amount of MSCs delivered will usually be greater when a solid, “patch” type application is made during an open procedure, but follow-up therapy by injection will be as described above. The frequency and duration of therapy will however, vary depending on the degree (percentage) of tissue involvement, as already described (e.g. 5-40% left ventricular mass).

In cases having in the 5-10% range of tissue involvement, it is possible to treat with as little as a single administration of one million cellular compositions in 20-50 μl of injection preparation. The injection medium can be any pharmaceutically acceptable isotonic liquid. Examples include phosphate buffered saline (PBS), culture media such as DMEM (preferably serum-free), physiological saline or 5% dextrose in water.

In cases having more in a range around the 20% tissue involvement severity level, multiple injections of 20-50 μl (10-40×10⁶ cells/ml) are envisioned, Follow-up therapy may involve additional dosings.

In very severe cases, e.g. in a range around the 40% tissue involvement severity level, multiple equivalent doses for a more extended duration with long term (up to several months) maintenance dose aftercare may well be indicated.

In another embodiment, the isolated and culture expanded cells (e.g. adult myocytes) or 3D-tissues can be utilized for the implantation of various prosthetic devices. For example, using porous ceramic structures filled with culture-expanded human mesenchymal stem cells, and implanting these structures in areas where there is extensive tissue damage.

The ability of MSCs to enhance growth and provide stability to myocyte cells provides an opportunity to employ tissues from genetically affected patients so as to create a 3-dimensional tissue construct specific to an individual or groups of individuals.

The constructs could also be used for therapies requiring tissue engineered material. Examples would be congenital heart disease, heart failure, MI, LVD.

Cardiotropic Agents: In some aspects of the invention, one or more “cardiotropic factors” can be included in the culture medium if it is desired by a user to differentiate the stem cells. These are factors that either alone or in combination enhance proliferation or survival of cardiomyocyte type cells, or inhibit the growth of other cell types. The effect may be due to a direct effect on the cell itself, or due to an effect on another cell type, which in turn enhances cardiomyocyte formation. For example, factors that induce the formation of hypoblast or epiblast equivalent cells, or cause these cells to produce their own cardiac promoting elements, all come within the rubric of cardiotropic factors.

Factors thought to induce differentiation of pluripotent stern cells into cells of the mesoderm layer, or facilitate further differentiation into cardiomyocyte lineage cells include the following: Nucleotide analogs that affect DNA methylation and altering expression of cardiomyoeyte-related genes TGF-β ligands (exemplified by TGF-β1, TGF-β2, TGF-β3 and other members of the TGF-β superfamily). Ligands bind a TGF-β receptor activate Type I and Type II serine kinases and cause phosphorylation of the Smad effector. Morphogens like Activin A and Activin B (members of the TGF-μ superfamily); Insulin-like growth factors (such as IGF II); Bone morphogenic proteins (members of the TGF-μ superfamily, exemplified by BMP-2 and BMF-4); Fibroblast growth factors (exemplified by bFGF, FGF-4, and FGF-8) and other ligands that activate cytosollc kinase raf-1 and mitogen-activated proteins kinase (MARK); Platelet-derived growth, factor (exemplified by PDGFβ) Natriuretic factors (exemplified by atrial natriuretic factor (ANF), brain natriuretic peptide (BMP). Related factors such as insulin, leukemia Inhibitory factor (LIF), epidermal growth factor (EGF), TGFα, and products of the cripto gene. Specific antibodies with agonist activity for the same receptors. Alternatively or in addition, the cells can be cocultured with cells (such as endothelial cells of various kinds) that secrete factors enhancing cardiomyocyte differentiation. Nucleotide analogs that affect DNA methylation (and thereby influence gene expression) can effectively be used to increase the proportion of cardiomyocyte lineage cells that emerge following initial differentiation. For example, it has been found that inclusion of 5-axα-deoxy-cytidine in the culture medium increases the frequency of contracting cells in the population, and expression of cardiac αMHC.

In preferred embodiments the factor comprises connexin-43 nucleic acids, peptides, polypeptides, variants mutants or active fragments thereof.

The combinations and amounts of compounds that are effective for enriching cardiomyocyte production can be determined empirically by culturing undifferentiated or early differentiated embryonic stem cells or their progeny in a culture environment incorporating such factors, and then determining whether the compound has increased the number of cardiomyocyte lineage cells in the population according to the phenotypic markers listed below.

Following initial differentiation (and before or after a separation step, if employed), it is possible to increase the percentage of cardiomyocyte lineage cells by culturing in an environment containing a “cardiomyoeyte enrichment agent”. This is a factor In the medium or on a surface substrate that promotes the outgrowth of the desired cell type—either by facilitating proliferation of cardiomyoeyte lineage cells, or by inhibiting the growth (or causing apoptosis) of cells of other tissue types. Some of the cardiotropic factors listed above are suitable for this purpose. Also suitable are certain compounds known beneficial to cardiomyocytes in vivo, or their analogs. Included are compounds capable of forming a high energy phosphate bond (such as creatine); an acyl group carrier molecule (such as carnitine); and a cardiomyoeyte calcium channel modulator (such, as taurine).

Cardiomyoeyte specific markers comprise: Cardiac troponin I (cTnI), a subunit of troponin complex that provides a calcium-sensitive molecular switch for the regulation of striated muscle contraction. Cardiac troponin T (cTnT) Atrial natriuretic factor (ANF), a hormone expressed in developing heart and fetal cardiomyocytes but down-regulated In adults. It is considered a good marker for cardiomyocytes because it is expressed in a highly specific manner in cardiac cells but not skeletal myocytes. The cells will also typically express at least one (and often at least 3, 5, or more) of the following markers: sarcomeric myosin heavy chain (MHC) Titin, tropomyosin, α-actinim and desmin, GATA-4, a transcription factor that is highly expressed in cardiac mesoderm and persists in the developing heart. It regulates many cardiac genes and plays a role in cardiogenesis Nkx2.5, a cardiac transcription factor expressed in cardiac mesoderm during early mouse embryonic development which persists in the developing bean. MEF-2A, MBP-2B, MEF-2C, MEF-2D; transcription factors that are expressed in cardiac mesoderm and persist in developing heart N-eadherin, which mediates adhesion among cardiac cells Connexin 43, which forms the gap junction between cardiomyocytes, β1-adrenoceptor (β1-AR) creatine kinase MB (CK-MB) and myoglobin, which are elevated in serum following myocardial infarction. Other markers that may be positive on cardiomyocytes and their precursors include α-cardiac actio, early growth response-1, and cyciin D2.

Tissue-specific markers can be detected using any suitable immunological technique-such as flow immunocytochemistry or affinity adsorption for cell-surface markers, immunocytochemistry (for example, of fixed cells or tissue sections) for intracellular or cell-surface markers. Western blot analysis of cellular extracts, and enzyme-finked immunoassay, for cellular extracts or products secreted into the medium. Expression of an antigen, by a cell is said to be antibody-detectable if a significantly detectable amount of antibody will bind to the antigen in a standard immunocytochemistry or flow cytometry assay, optionally alter fixation of the cells, and optionally using a labeled secondary antibody or other conjugate (such as abiotin-avidin conjugate) to amplify labeling.

The expression of tissue-specific gene products can also be detected at the mRNA level by Northern blot analysis, dot-blot hybridization analysis, or by reverse transcriptase initiated polymerase chain reaction (RT-PCR) using sequence-specific primers in standard, amplification methods. See U.S. Pat. No. 5,843,780 for details of general technique. Sequence data for other markers listed in this disclosure can be obtained from public databases such as GenBank (URL www.ncbi.nlm.nih.gov:80/entrez). Expression at the mRNA level is said to be detectable according to one of the assays described in this disclosure if the performance of the assay on cell samples according to standard procedures in a typical controlled experiment results in clearly discemable hybridization or amplification product. Expression of tissue-specific markers as detected at the protein or mRNA level is considered positive if the level is at least 2-fold, and preferably more than 10- or 50-fold above that of a control cell, such as an undifferentiated pluripotent stem cell or other unrelated cell type.

Once markers have been identified on the surface of cells of the desired phenotype, they can be used for Immunoselection to further enrich the population by techniques such as immunopanning or antibody-medicated fluorescence-activated cell sorting,

Gene Therapy: In some preferred embodiments, the compositions comprise therapeutic genes for delivery into the body, such as for example, the heart. In sense embodiments, the therapeutic gene is a transgene. For example, the delivery cells—e.g. the mesenchymal stem cells are removed from the body, and a therapeutic transgene is introduced into them via vehicles well known to those skilled in the art such as those used in direct-gene-transfer methods. For example, while still in the laboratory, the genetically modified cells are tested and then allowed to grow and multiply and, finally, are infused back into the patient. Alternatively, allogeneic cells that, bear normal, endogenous genes can reverse a deficiency in a particular target tissue. Use of cells bearing either transgenes or normal, endogenous genes is referred to herein as gene therapy.

The cells can also be genetically altered in order to enhance their ability to be involved in tissue regeneration, or to deliver a therapeutic gene to a site of administration. A vector is designed using the known encoding sequence for the desired gene, operatively linked to a promoter that is either pan-specific or specifically active in the differentiated cell type. Of particular interest are cells that are genetically altered to express one or more growth factors of various types, cardiotropic factors such as atrial natriuretic factor, cripto, and cardiac transcription regulation factors, such as GATA-4, Nkx2.5. and MEF2-C. Production of these factors at the site of administration may facilitate adoption of the functional phenotype enhance the beneficial effect of the administered cell, or increase proliferation or activity of host cells neighboring the treatment site. In preferred embodiments, the molecule is connexin-43 as described above.

Implantation of ex-vivo Tissue Constructs: The transplantation of ex-vivo tissue constructs into the myocardium of a subject can use well known surgical techniques for grafting tissue and/or isolated cells into a heart. In general, there are two methods for introducing the ex-vivo myocardial tissue constructs into the subject's heart tissue; 1) surgical, direct injection; or 2) percutaneous techniques as describe in U.S. Pat. No. 6,059,726 (Lee and Lesh, “Method for locating the AV junction of the heart and injecting active substances therein”).

The ex-vivo tissue constructs can be implanted into any area of the heart where conduction disturbances have occurred. The amount and location of ex-vivo tissue constructs to be transplanted is determined by the type of heart disease being treated and the overall damage of myocardial tissue.

In certain embodiments, the ex-vivo tissue constructs are transplanted by percutaneous methods. If the site of the damaged heart tissue can be accurately determined in a subject by non-invasive diagnostic techniques, the ex-vivo myocardial tissue constructs can be injected directly into the damaged myocardial tissue using general methods for percutaneous injections into cardiac muscle well known in the art. The amount of ex-vivo myocardial tissue constructs necessary to be therapeutically effective will vary with the type of disorder being treated as well as the extent of heart damage that has occurred.

Immunosuppressants may be used in conjunction of transplantation of ex-vivo

myocardial tissue constructs not derived from the host to minimize the possibility of graft rejection, e.g., allogeneic or xenogeneic cells.

### Drug Screening

Cell and tissue compositions of this invention can be used to screen for factors (such as solvents, small molecule drugs, peptides, oligonucleotides) or environmental conditions (such as culture conditions or manipulation) that affect the characteristics of such cells and their various progeny.

In some applications, the compositions are used to screen factors that promote maturation into later-stage cardiomyocyte precursors, or terminally differentiated cells, or to promote proliferation and maintenance of such cells in long-term culture. For example, candidate maturation factors or growth factors are tested by adding them to cells in different wells, and then determining any phenotypic change that results, according to desirable criteria for further culture and use of the cells.

Other screening applications of this invention relate to the testing of pharmaceutical compounds for their effect on cardiac muscle tissue maintenance or repair. Screening may be done either because the compound is designed to have a pharmacological effect on the cells, or because a compound designed to have effects elsewhere may have unintended side effects on cells of this tissue type. The screening can be conducted using any of the stem cell, tissues or terminally differentiated cells.

The reader is referred generally to the standard textbook In vitro Methods in Pharmaceutical Research, Academic Press, 1997, and U.S. Pat. No. 5,030,015. Assessment of the activity of candidate pharmaceutical, compounds generally involves combining the cells of this invention with the candidate compound, either alone or in combination with other drugs. The investigator determines any change in the morphology, marker phenotype, or functional activity of the cells that is attributable to the compound (compared with untreated cells or cells treated with an inert compound), and then correlates the effect of the compound with the observed change.

Cytotoxicity can be determined in the first instance by the effect on cell viability, survival, morphology, and the expression of certain markers and receptors. Effects of a drug on chromosomal DNA can be determined by measuring DNA synthesis or repair. [³H]-thymidine or BrdU incorporation, especially at unscheduled times in the cell cycle, or above the level required for cell, replication, is consistent with a drug effect. Unwanted effects can also include unusual rates of sister chromatid exchange, determined by metaphase spread. The reader is referred to A. Vickers (pp 375-410 in In vitro Methods in Pharmaceutical Research, Academic Press, 1997) for further elaboration.

Effect of cell function can be assessed using any standard assay to observe phenotype or activity of cardiomyocytes such as marker expression, receptor binding, contractile activity, or electrophysiology—either in cell culture or in vivo. Pharmaceutical candidates can also be tested for their effect on contractile activity—such as whether they increase or decrease the extent or frequency of contraction. Where an effect is observed, the concentration of the compound can be titrated to determine the median effective dose (ED₅₀).

### Treatment

The amount of cells administered to the patient will, also vary depending on the condition of the patient and should be determined via consideration of all appropriate factors by the practitioner. Preferably, however, about 1×10⁶ to about 1×10¹² more preferably about 1×10⁸ to about 1×10¹¹, more preferably, about 1×10⁹ to about 1×10¹⁰ cells are utilized for adult humans. These-amounts will vary depending on the age, weight, size, condition, sex of the patient, the type of tumor to be treated, the route of administration, whether the treatment is regional or systemic, and other factors. Those skilled in the art should be readily able to derive appropriate dosages and schedules of administration to suit the specific circumstance and needs of the patient.

Methods of re-introducing cellular components are known in the art and include procedures such as those exemplified in U.S. Pat. No. 4,844,893 to Honsik, et al. and U.S. Pat. No. 4,690,915 to Rosenberg.

### Pharmaceutical Compositions

In other embodiments, the present invention provides pharmaceutical compositions comprising the cellular and/or tissue compositions. In other aspects, the present invention features kits for treating cardiac tissue damage or for delivering a functional gene or gene product to the heart in a mammal comprising the cells and/or 3D-tissues. Cells generally have been presented to the desired organs either by injection into the tissue, by infusion into the local circulation, or by mobilization of autologous stem cells from the marrow accompanied by prior removal of stem cell-entrapping organs before mobilization when feasible, i.e., splenectomy.

In some embodiments, the administration of the compositions can be coupled with other therapies. For example, a therapeutic agent can be administered prior to, concomitantly with, or after infusing the cells to a patient.

Administration of cells transduced ex vivo can be by any of the routes normally used for introducing a cell or molecule into ultimate contact with blood or tissue cells. The cells or tissues may be administered in any suitable manner, preferably with pharmaceutically acceptable carriers, in the case of the tissues, the 3D-tissues can be implanted via surgical methods or intra-cardial injections. Suitable methods of administering cells or tissues in the context of the present, invention to a patient are available, and, although more than one route can be used to administer a particular composition, a particular route can often provide a more immediate and more effective reaction than another route.

Pharmaceutically acceptable carriers are determined in part by the particular composition being administered, as well as by the particular method used to administer the composition. Accordingly, there is a wide variety of suitable formulations of pharmaceutical compositions of the present invention.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Parenteral administration is one useful method of administration. The formulations can be presented in unit-dose or multi-dose sealed containers, such as ampoules and vials, and in some embodiments, can be stored in a freeze-dried (lyophilized) condition requiring only the addition of the sterile liquid carrier, for example, water, for injections, immediately prior to use. These formulations may be administered with factors that mobilize the desired class of adult stem cells into the circulation.

Extemporaneous injection solutions and suspensions can be prepared from sterile powders, granules, and tablets of the kind previously described. Cells transduced by the vector as described above in the context of ax vivo therapy can also be administered parenteraliy as described above, except that lyophilization is not generally appropriate, since cells are destroyed by lyophilization. The close administered to a patient, in the context of the present invention should be sufficient to effect a beneficial therapeutic response in the patient over time. The dose will be determined by the efficacy of the particular cells employed and the condition of the patient, as well as the body weight of the patient to be treated. The size of the dose also will be determined by the existence, nature, and extent of any adverse side effects that accompany the administration of a cell type in a particular patient, in determining the effective amount of cells to be administered in the treatment or prophylaxis of diseases, the physician should evaluate circulating plasma levels, and, in the case of .replacement therapy, the production of the gene product of interest.

Transduced cells are prepared for re-infusion according to established methods. See, Abrahamsen et al., J. Clin. Apheresis 6:48-53(1991); Carter et al., J. Clin. Apheresis 4:113-117(1988); Aebersold et al., J. Immunol. Methods 112: 1-7(1988); Muul et al., J. Immunol. Methods 101:171-181(1987) and Carter et al., Transfusion 27:362-365 (1987). After a period of about 2-4 weeks in culture, the cells may number between 1×10⁶ and 1×10¹⁰. In this regard, the growth characteristics of cells vary from patient to patient and from cell type to cell type. About 72 hours prior to reinfusion of the transduced cells, an aliquot is taken for analysis of phenotype, and percentage of cells expressing the therapeutic agent.

For administration, cells of the present invention can be administered at a rate determined by the ID₅₀ of the cell type, and the side effects of the cell type at various concentrations, as applied to the mass and overall health of the patient. Administration can be accomplished via single or divided doses. Adult stem cells may also be mobilized using exogenously administered factors that stimulate their production and egress from tissues or spaces, that may include, but are not restricted to, bone marrow or adipose tissues.

The invention has been described in detail with reference to preferred embodiments thereof. However, it will be appreciated that those skilled in the art, upon consideration of this disclosure, may make modifications and improvements within the spirit and scope of the invention. The following non-limiting examples are illustrative of the invention.

All documents mentioned herein are incorporated herein by reference. All publications and patent documents cited in this application are incorporated by reference for all purposes to the same extent as if each individual publication or patent document were so individually denoted. By their citation of various references in this document. Applicants do not admit any particular reference is “prior art” to their invention.

### EXAMPLES

While various embodiments of the present invention have been described above, it should be understood that they have been presented by way of example only, and not limitation. Numerous changes to the disclosed embodiments can be made in accordance with the disclosure herein without departing from the spirit or scope of the invention. Thus, the breadth and scope of the present invention should not be limited by any of the above described embodiments.

Materials and Methods

Lentiviral vectors were created to knockdown (KD) Cx43 and to overexpress (OE) Cx43 in human MSCs.

Human MSCs with altered expression of Cx43 (Cx43 KD, Cx43 OE and controls) were co-cultured with NRVMs at the ratio 1:5 during 4 weeks.

Results

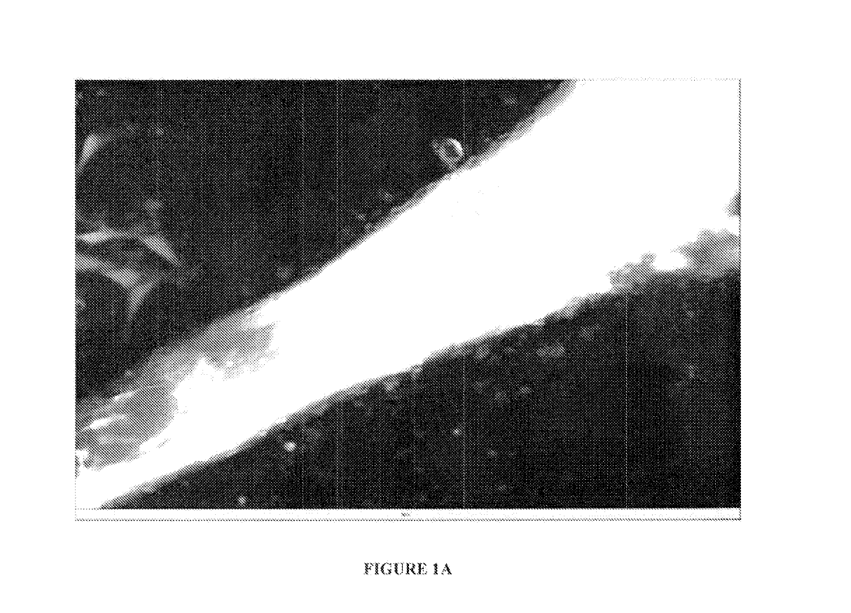

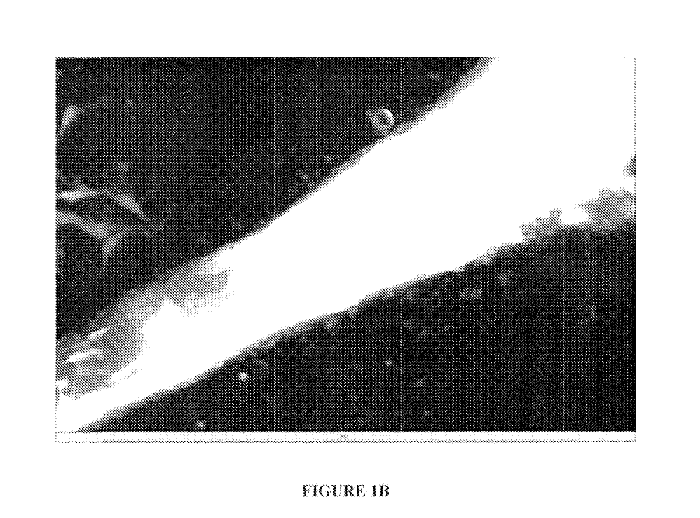

FIGS. 1A and 1B show that human MSCs (GFP⁺) in co-culture with NRVMs form beating tubes. GFP⁺ cells integrate into the tube.

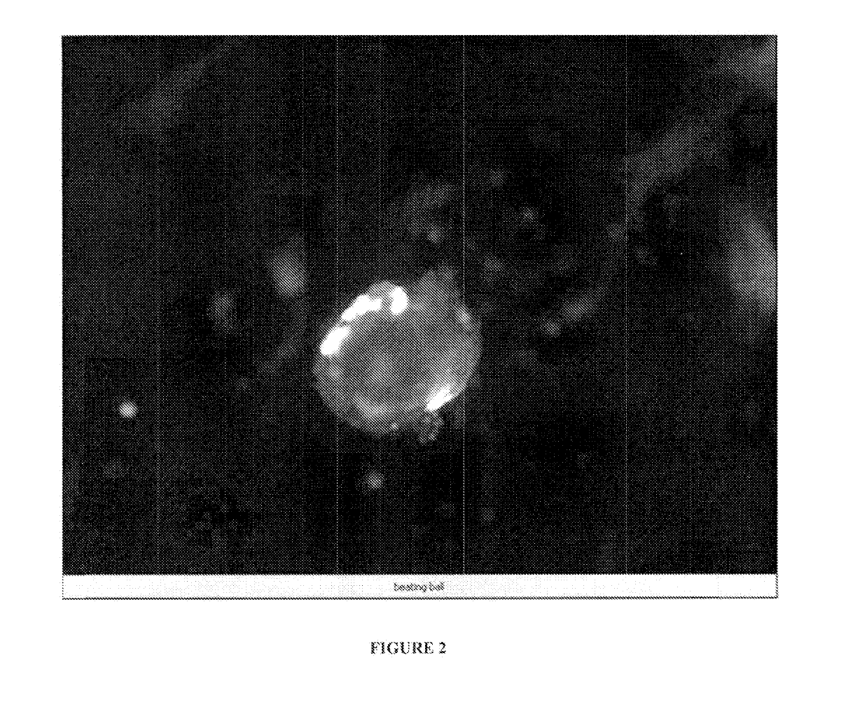

FIG. 2 shows a beating sphere in co-culture CX43 knockdown hMSCs with NRVMs.

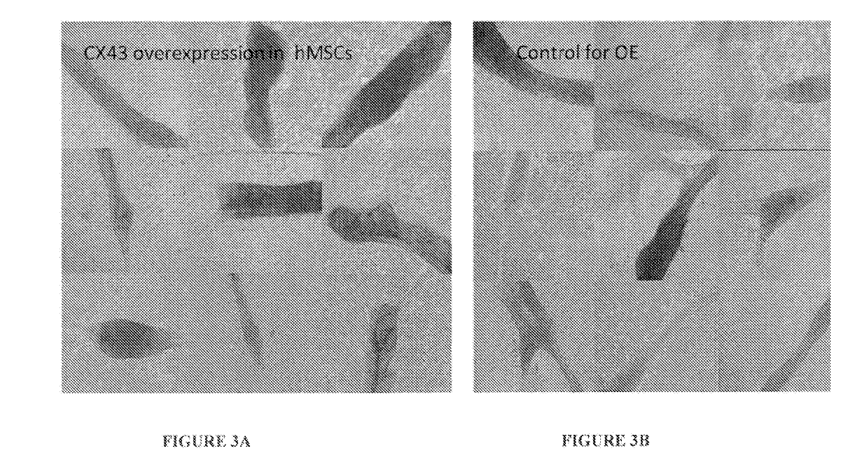

FIG. 3A shows that beating tube formation occurred after 10 days in co-culture NRVMs with hMSCs overexpressing Cx43. FIG. 3B shows the control group where tube formation, occurred after 14 days in co-culture.

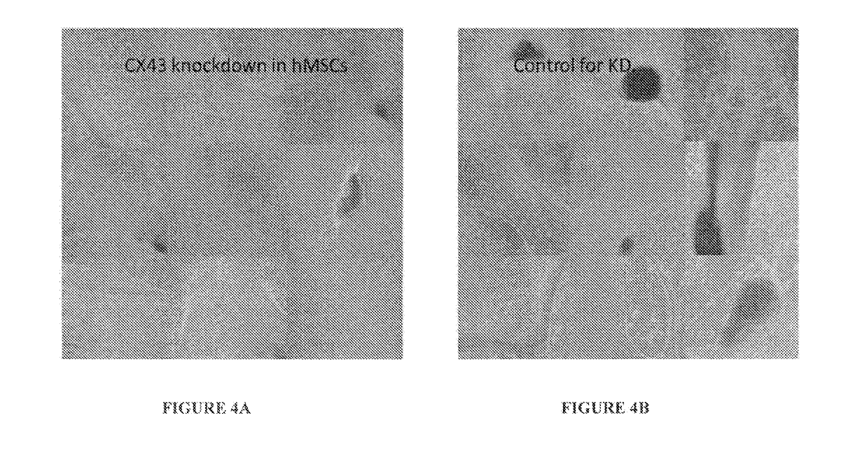

FIG. 4A shows that in co-cultured hMSCs Cx43 knockdown, with NRVMs beating tube formation never occurred. FIG. 4B shows that in the control group beating tube formation occurred after 14 days in co-culture.

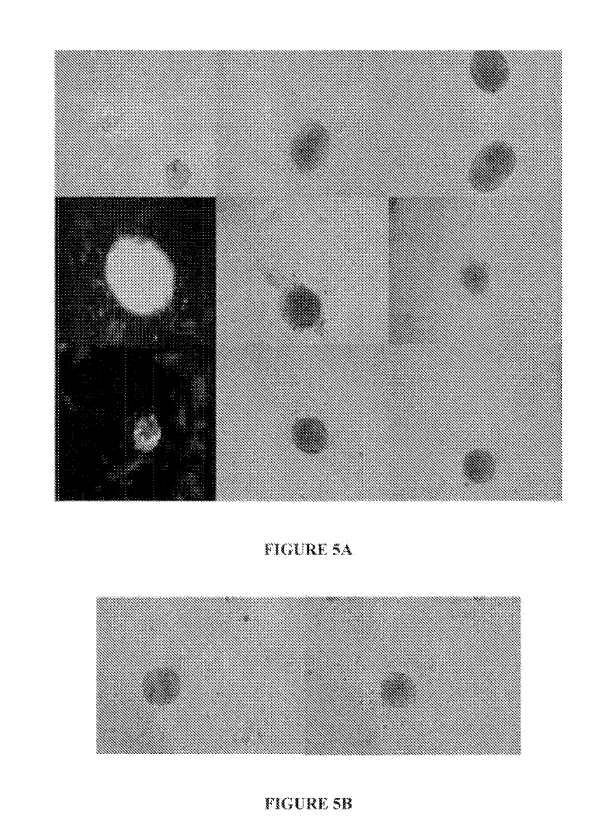

FIG. 5A shows that beating spheres occurred in NRVMs with hMSC Cx43 knockdown co-culture after 4 weeks. FIG. 5B shows that NRVMs alone form beating spheres in culture.

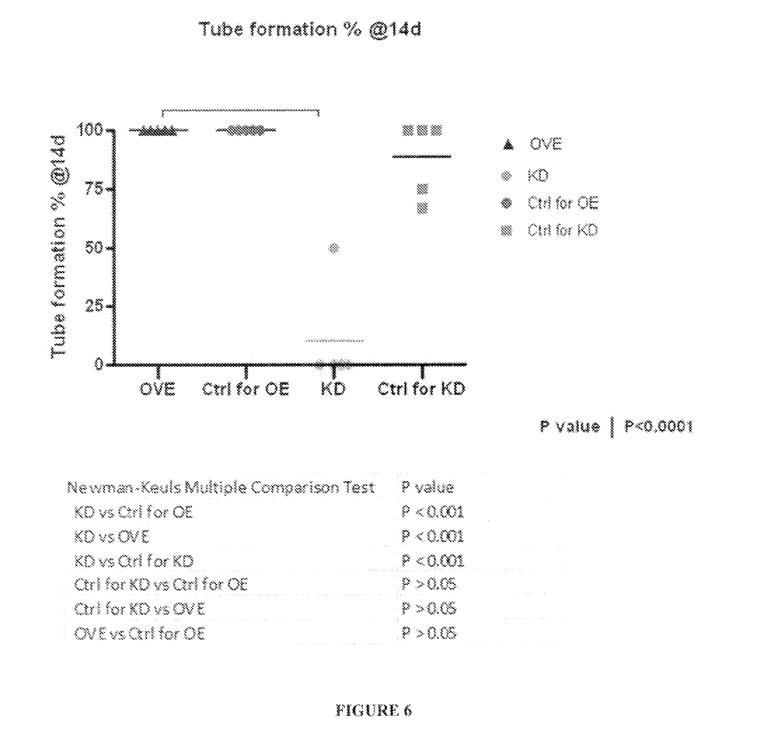

FIG. 6 is a graph showing the percentage of tube formation at 14 days.

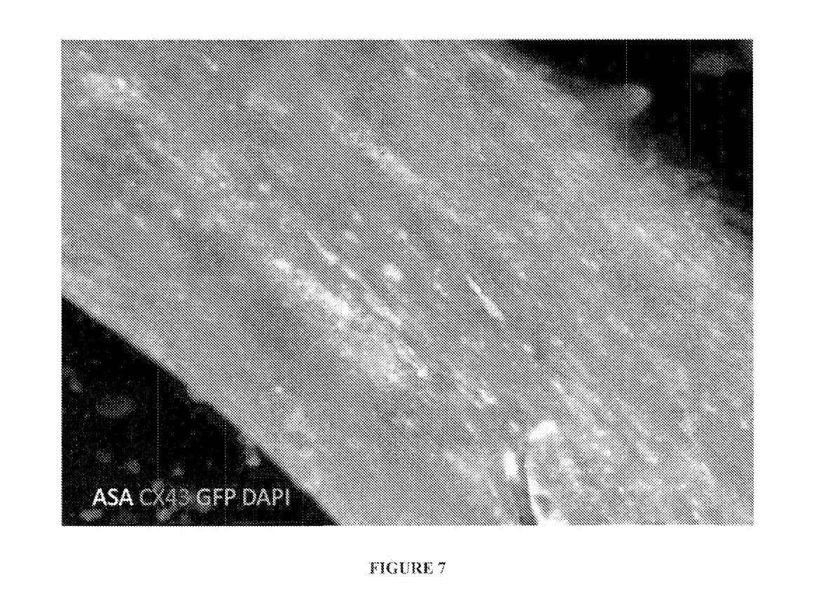

FIG. 7 shows beating tube formation of NRVMs with human MSCs overexpressing Cx43.

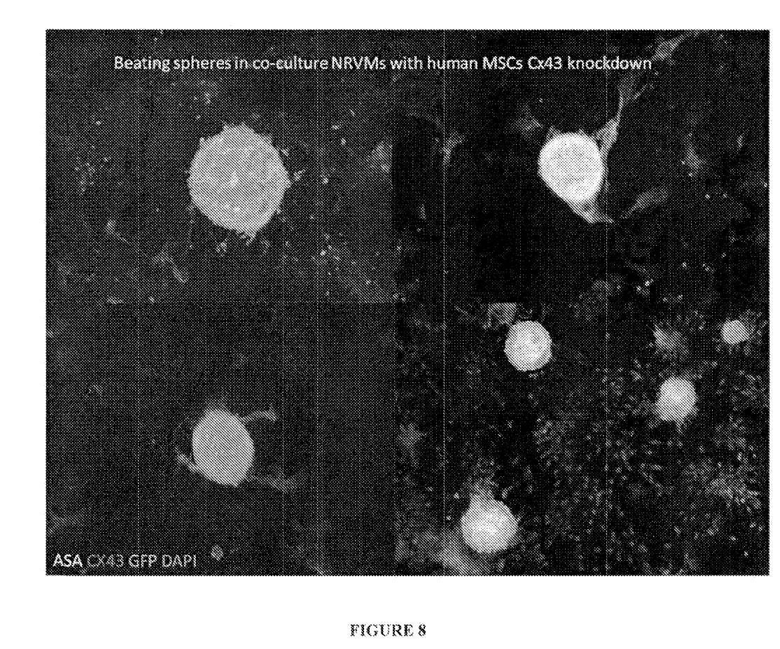

FIG. 8 shows beating spheres in co-culture NRVMs with human MSCs Cx43 knockdown.

What is claimed is:

1. A method of engineering a three-dimensional tissue comprising; co-culturing an effective amount of isolated mesenchymal stem cells and myocytes; and,
engineering a three-dimensional tissue.

2. The method of claim 1, wherein the mesenchymal stem cells and myocytes are autologous, heterologous, syngeneic, allogeneic or xenogeneic cells.

3. The method of claim 1, wherein the mesenchymal stem, cells are multi-lineage stem cells.

4. The method of claim 1, wherein the myocytes comprise cardiac stem cells, neonatal myocytes, adult myocytes or combinations thereof.

5. The method of claim 1, wherein the co-culture of mesenchymal stem cells and myocytes differentiate and form a three-dimensional beating myocardial tissue.

6. A co-culture system comprising isolated mesenchymal stem cells, tissues comprising mesenchymal stem cells, myocytes, tissues comprising myocytes or combinations thereof.

7. The co-culture system of claim 6, wherein the mesenchymal stem cells, myocytes or tissues are autologous, heterologous, syngeneic, allogeneic or xenogeneic cells or tissues.

8. The co-culture system of claim 6, wherein the mesenchymal stem cells are multi-lineage stem cells.

9. The co-culture system of claim 6, wherein the myocytes comprise cardiac stem cells, neonatal myocytes, adult myocytes or combinations thereof.

10. The co-culture system of claim 6, wherein the co-culture of mesenchymal stem cells and myocytes differentiate and form a three-dimensional beating myocardial tissue.

11. A method of culturing adult myocytes comprising: co-culturing an effective amount of isolated mesenchymal stem cells and myocytes; and,
culturing adult myocytes.

12. The method of claim 11, wherein the mesenchymal stem cells and myocytes are autologous, heterologous, syngeneic, allogeneic or xenogeneic cells.

13. The method of claim 11, wherein the mesenchymal stem cells are multi-lineage stem cells.

14. The method of claim 11, wherein the adult myocytes are maintained and expanded in vitro.

15. A method of treating or repairing damaged cardiac tissue comprising administering to a patient in need thereof, myocytes which have been cultured ex vivo and/or grafting into a patient in need thereof, a three-dimensional tissue graft; and,
treating damaged cardiac tissue.

16. The method of claim 15, wherein cultured myocytes are obtained from a co-culture of mesenchymal stem cells and myocyte cells.

17. The method of claim 15, wherein a three-dimensional tissue graft is obtained by co-culturing an effective amount of isolated mesenchymal stem cells and myocytes.

18. The method of claims 15, wherein the mesenchymal stem cells and myocytes are autologous, heterologous, syngeneic, allogeneic or xenogeneic cells.

19. The method of claims 15, wherein the mesenchymal stem cells are multi-lineage stem cells.

20. The method of claims 15, wherein the myocytes comprise cardiac stem cells, neonatal myocytes, adult myocytes or combinations thereof.

21. The method of claim 15, wherein the mesenchymal stem cells comprise connexin molecules.

22. The method of claim 15, wherein the mesenchymal stem cells lack connexin molecules.

23. A method of engineering a three-dimensional tissue comprising: co-culturing an effective amount of isolated mesenchymal stem cells and myocytes; or isolated mesenchymal stem cells, tissues comprising mesenchymal stem cells, myocytes, tissues comprising myocytes or combinations thereof.

24. The method of claim 23, wherein the mesenchymal stem cells, myocytes or tissues are autologous, heterologous, syngeneic, allogeneic or xenogeneic cells or tissues.

25. The method of claim 23, wherein the mesenchymal stem cells are multi-lineage stem cells.

26. The method of claim 23, wherein the myocytes comprise cardiac stem cells, neonatal myocytes, adult myocytes or combinations thereof.

27. The method of claim 23, wherein the co-culture of mesenchymal stem cells and myocytes differentiate and form a three-dimensional beating myocardial tissue.
